(19) United States
(12) Patent Application Publication
(10) Pub. No.: US 2026/0235494 A1
(43) Pub. Date: Aug. 13, 2026

(54) FRICTION TESTING APPARATUS AND METHOD

(71) Applicant: PCS INSTRUMENTS LTD, London (GB)

(72) Inventors: Clive Hamer, London (GB); Matt Smeeth, London (GB)

(21) Appl. No.: 19/154,556

(22) PCT Filed: Apr. 16, 2024
(86) PCT No.: PCT/EP2024/060312

§ 371 Date: Aug. 7, 2025

(30) Foreign Application Priority Data
May 20, 2023 (GB) 2307582.3

(51) Int. Cl. G01N 19/02

(52) U.S. Cl. CPC G01N 19/02

(57) ABSTRACT

The invention relates to a test apparatus for measuring frictional properties of a fluid or of a specimen immersed in a fluid. A first specimen holder holds a first specimen in the fluid being tested; the first specimen in contact with a surface of a second specimen, in a second specimen holder. A measurable load is applied between the two specimens, and an oscillatory driving means oscillates the first specimen holder relative to the second specimen holder. Force measuring means measures the frictional force between the two specimens. The specimen holders are in a sealed pressure chamber allowing the test to be carried out under alternative pressures. A push rod provides the oscillating motion of the first specimen holder inside the pressure chamber so the pressure is maintained during the test. The pressure chamber is removable and maintains pressure or vacuum even when removed from the test apparatus.

### BACKGROUND

This invention relates to apparatus and method for testing the lubricating properties of liquids, gases or other fluids and/or the frictional and wear properties of materials. Such apparatus simulates the conditions of operation of a mechanical device having moving parts and a fluid lubricant, such as for example a fuel pump or a gear box. Measurements can be made of the condition of the lubricant, or other fluid under test, the condition of the moving parts and the forces acting on the moving parts during the test. From these measurements it is typically possible to test new materials and lubricants or other fluids before sale as new products or before introduction into new products as components.

An important feature of such testing machines is that they reproduce reliable results for a range of test fluids in different environments. The results are dependent on the reliability of the wear on the test sample which needs to be accurately correlated with the properties of the test fluid in the conditions that the test fluid would experience during its proposed use.

A previous disclosure UK U.S. Pat. No. 2,270,387 describes an apparatus for testing lubricity using a rigid push rod.

It is an objective of the present invention to provide an improved testing apparatus which tests the fluid and/or the materials in conditions which are closer to the real conditions to be experienced by the fluid or materials.

### STATEMENT OF INVENTION

According to the invention there is provided a test apparatus and method according to the appended claims.

### DESCRIPTION OF FIGURES

The invention is illustrated, merely by way of example, in the accompanying drawings in which:

FIG. 1 is a perspective view of the of the testing apparatus in accordance with a first embodiment of the invention,

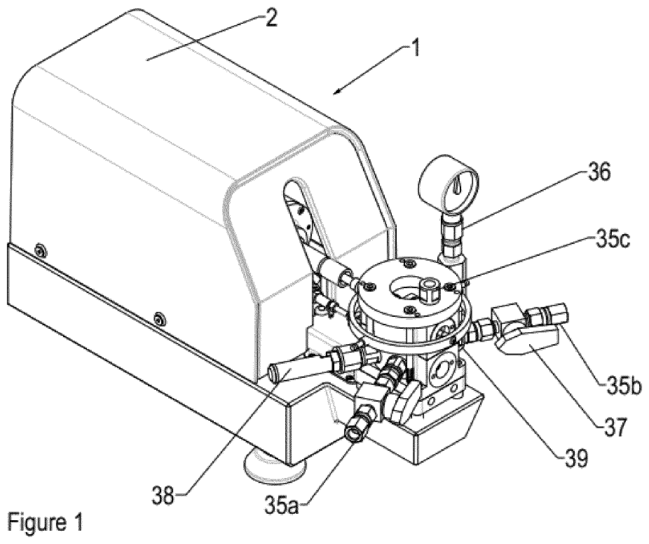

FIG. 2 is a side view of the front portion of the testing apparatus of FIG. 1 where the test parts engage,

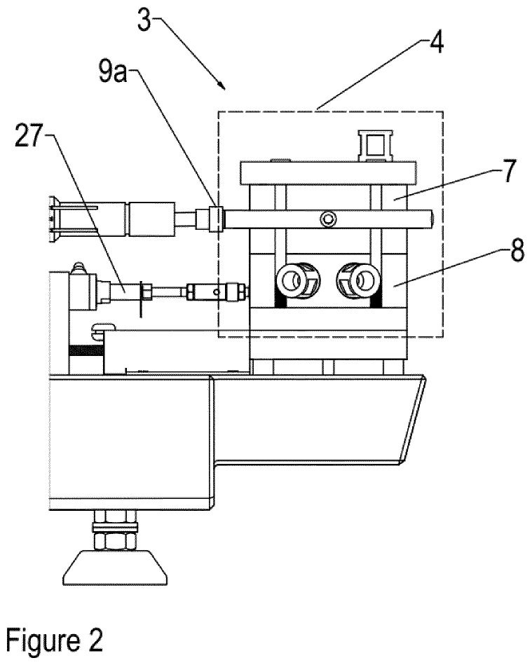

FIG. 3 is a front end view of the testing apparatus of FIG. 1,

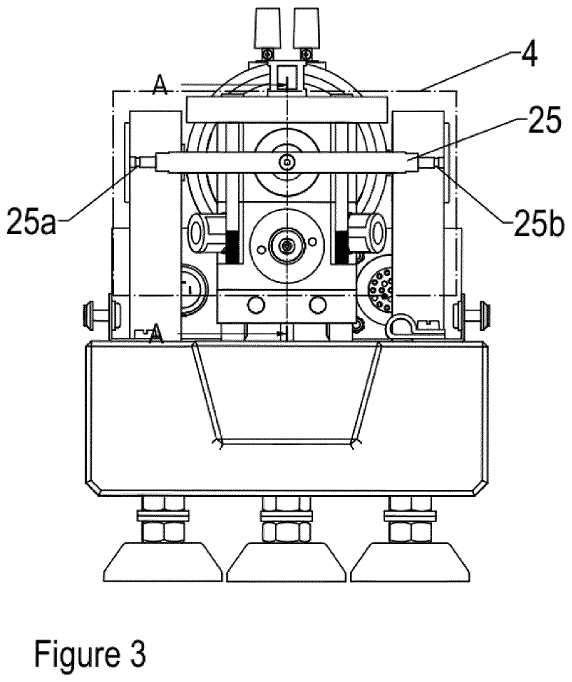

FIG. 4 is an enlarged perspective view of the front portion of the testing apparatus of FIG. 1,

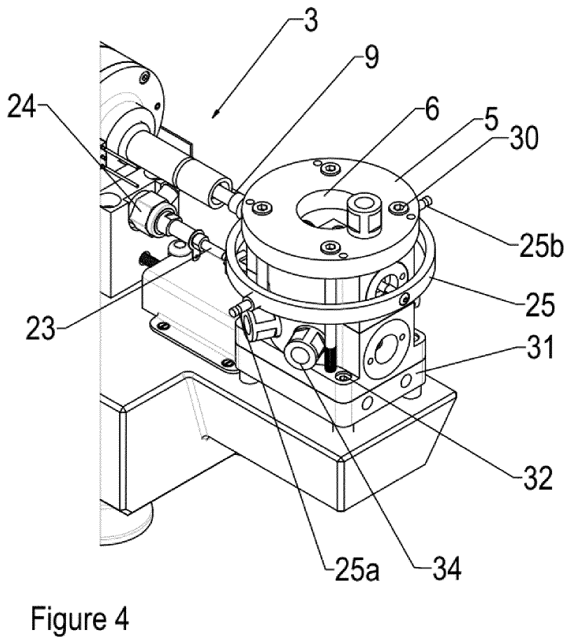

FIG. 5 is a schematic side cross sectional view of the front portion of the testing apparatus of FIG. 1, and

FIG. 6 is a perspective view of the front portion of the testing apparatus separated from the testing apparatus.

### DETAILED DESCRIPTION:

Referring to the drawings, there is shown one main embodiment of a test apparatus 1 according to the present invention including a housing 2 and a front portion 3 in which upper and lower specimens 13 and 14 may be mounted. The upper specimen 13 is releasedly held in the upper specimen holder 10 by a grub screw 12. The upper specimen 13 comprises a sphere of the material under test. It will be appreciated that in other embodiments the lower specimen could be the material under test and or the fluid in contact with the upper and lower specimens 13, 14. The upper specimen holder 10 is attached by means of a support block 11 to a push rod 9 which in turn is connected to the electromagnetic vibrator (not shown) and located inside the housing 2. The test involves oscillating the test ball, being the upper specimen 13, under load against the lower specimen 14 which is in the form of a test disc.

Following each test the wear indication or wear scar on either or both of the upper specimen 13 and lower specimen 14 is measured in width, length and depth dimensions and this correlates with the wear resistant properties of either specimen and/or the frictional properties of the fluid being tested.

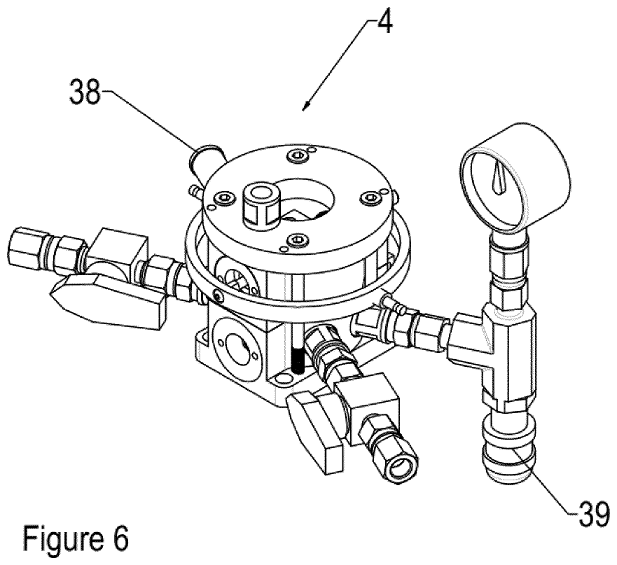

In this embodiment a fully sealed pressure chamber 4 is provided around the contact area of the lower and upper specimens 13, 14, which is enables a test to be run under high-pressure or vacuum. The front portion 3 of the device is removable and includes the pressure chamber 4 which maintains pressure or vacuum even when removed from the test apparatus 1 to another location so that test blends can be made away from the remainder of the test apparatus 1. FIG. 6 shows the separated front portion 3 for pressurisation in a location.

A test may be carried out on a fuel or lubricant under the same pressure and temperature conditions as would be experience during use and so a fast, reliable and inexpensive way to test fuels and lubricants, or parts in contact with fuels or lubricants, is achievable.

The pressure chamber 4 is enclosed by three parts, a removable lid 5 with a glass window 6, an upper chamber part 7 and a lower chamber part 8.

The lower specimen holder 14 comprises an upstanding wall 16 which contains the test fluid 17 if required.

The upper specimen holder 10 is located in an upper support block 11, which is attached to two metal bellows 18a, 18b, which extend from each side of the support block 11 and are attached to internal wall 19 of the upper chamber part 7. The bellows 18a, 18b are flexible to allow a lateral oscillation and load to be applied, via the push rod 9 to the ball-shaped upper specimen 13 to the disc-shaped lower specimen 14, and can maintain a sealed pressure chamber 4 so that a pressurized gas or vacuum can be maintained in the chamber 4. The push rod 9 is coupled, by means of decouplable push rod coupling 9a to an external vibrator (not shown) located within the housing 2 which enables the ball-shaped upper specimen 13 to be driven in a reciprocating motion.

The push rod 9 is attached to diametrically opposite load bearing supports 25a, 25b by means of a support ring 25 so that weights can be hung from the load bearing supports 25a, 25b according to the load desired to be maintained between the specimens 13, 14 during a test.

The lower specimen holder 15 is attached to a lower support block 20 which forms part of the lower chamber part 8. Two thin discs 21a, 21b are attached to each of respective blocks ends 22a, 22b at the centre region of the discs 21a, 21b, and the discs 21a, 21b being fixed in the circumferential region of the discs 21a 21b to an inside surface of the lower chamber part 8. The discs 21a, 21b are axially flexible but stiff in the radial directions. The discs 21a, 21b act as a flexible seal to allow a pressure gas or vacuum to be maintained inside the chamber 4 on insides of the discs 21a, 21b against the ambient pressure outside the chamber 4 on the outside of the discs 21a, 21b. The lower support block 20 is also attached, by a pushrod 23, to a high stiffness force transducer 24. The relative stiffness of the force transducer 24 to flexible discs 21a, 21b means that a high proportion of the friction force is applied to the force transducer and as the combined discs 21a, 21b and force transducer 24 arrangement is fully elastic the signal from the force transducer 24 is directly proportional to the friction force exerted on the lower specimen 14. As discussed above the lower specimen holder 15 is supported by two flexible discs 21a, 21b at either end. There is no other physical contact except between the two specimens 13, 14 so any friction force is transmitted through the two discs 21a, 21b. The disc 21a, 21b have a relatively low stiffness in the axial direction compared to the force transducer 24 that the lower specimen holder 15 is coupled to. The discs 21a, 21b act as a means of sealing the pressure chamber 4 such that a pressure of up to 10 bar or a vacuum in the pressure chamber can be maintained. If there is a vacuum in the pressure chamber 4 and there is no test fluid, heat can only be transferred to the lower specimen holder 15 through the discs 21a, 22b and their isolating pads 26a, 26b, acting as supports which need to be electrically insulating. Preferably the discs 21a, 21b are made from nickel plated beryllium copper (BeCu) and the isolating pad supports 26a, 26b are made from anodized aluminium-both very good thermal conductors and the anodizing of the aluminium provides electrical insulation.

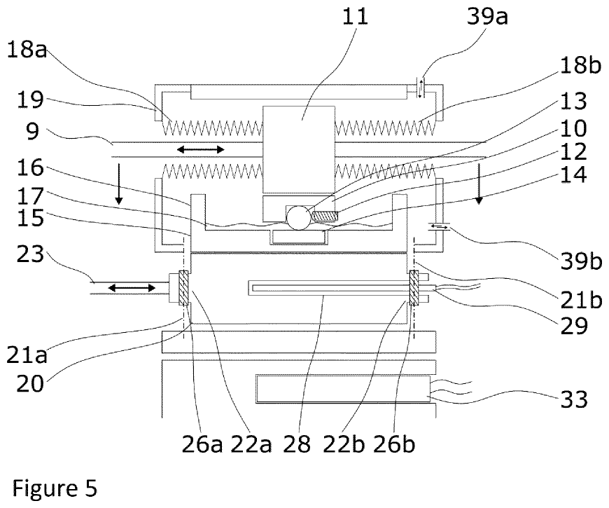

Refering to FIGS. 5 to 8, the lower chamber part 8 includes a pressure borehole 34 into which a pressure line 35 is fitted so that fluid under positve or negative pressure can be introcued or removed from the pressure chamber 4. There are three pressure lines 35a, 35b, 35c arrranged diametrically around the lower chamber part 8. The pressure line 35a includes a pressure gauge 36 as well as a proportional relief valve 39. Pressure lines 35a and 35b include actuation valves 37 for controlling the introduction or removal of fluid and also include a safety release valve 38.

The discs 21a, 21b are electrically insulated from the internal wall, such as by means of an isolating pads 26a, 26b and an electrically insulating coupling 27 is provided the between pushrod 23 and the force transducer 24 so that the whole traducer arrangement is electrically insulated. This allows an electrical current to be applied across the contact between the specimens to measure its resistance or to investigate the effects of different voltages or currents on the contact behaviour.

The lower specimen holder support block 20 includes a borehole 28 to facilitate the insertion of a temperature measurement probe 29.

The pot lid 5, upper chamber part 7 and lower chamber part 8 form the sealed chamber 4 locked in place by four long screws 30. This whole assembly is attached to a heater block 31 by four short screws 32. The heater block 31 contains cartridge heaters 33 to allow the chamber 4 to be heated and the temperature controlled by the input from the temperature measurement probe 29 inputted to a temperature controller 34.

By removing the four short screws 32, unclamping the force transducer coupling 27 and the pushrod coupling 9a, the whole pressure chamber test assembly 3 can be removed from the test machine 1 whilst maintaining the pressure in the chamber 4 so that test blends can made within the pressurized chamber 4 away from the remainder of the test machine 1.

The heater cartridges 34 in the temperature regulating block 31 may contain electric heaters or cooling elements which together with temperature measurement probe 29 and the controller to enable tests to be carried out at elevated temperatures or reduced temperatures. The temperature regulating block 16 is attached to a flexural support which is designed to be stiff in all directions except the direction of oscillation of the upper sample 13, in which direction it is allowed to deflect.

Suitably the lower specimen holder 15 is the form of a stainless steel bath to contain the test lubricant. The lower specimen holder 15 in the form of a bath comprises an upstanding wall 16 to which the lower specimen is clamped. The lower specimen holder 15 can contain a small quantity of test fluid or test grease or can be a dry contact and the remaining volume of the pressure chamber is filled with a pressurized gas, which may be a test gas, or put under vacuum.

Alternatively, the lower specimen holder can be a flat plate to which the lower specimen 14 is clamped. The whole lower half of the pressure chamber 4 can then be filled with the test fluid which submerges the test specimen contact and the volume above the test fluid is filled with a pressurized gas, which may be a test gas, or put under vacuum. The lower specimen 14, when clamped to the flat plate, may be coated with a test grease or can be a dry contact and the remaining volume of the pressure chamber filled with a pressurized gas, which may be a test gas, or put under vacuum.

By means of this apparatus it is also possible to measure the electrical resistance of the contact between the two specimens. This resistance is determined by the degree of asperity to asperity contact between the specimens and is a qualitative measure of the effectiveness of the lubricant at separating the specimens.

During these measurements it is possible to vary the parameters of load and specimen temperature and pressure by the methods described above.

### COMPONENT LIST

1—Testing apparatus
2—Housing
3—Front portion
4—Pressure chamber
5—Removable Lid
6—Glass window
7—Upper chamber part
8—Lower chamber part
9—Push Rod
9a—Push rod coupling
10—Upper Specimen Holder/first specimen holder
11—Support block
12—Grub Screw
13—Upper Specimen/first specimen
14—Lower Specimen/second specimen
15—Lower Specimen Holder/second specimen holder
16—Upstanding wall
17—Test fluid
18a, 18b—Bellows
19—Internal wall
20—Lower support block
21a, 21b—Discs
22a, 22b—Block ends
23—Push rod
24—Force transducer
25—Support ring
25a, 25b—Load bearing supports
26a, 26b—Isolating pads
27—Insulated coupling
28—Borehole
29—Temperature measurement probe
30—Long screws
31—Heater block
32—Short screws
33—Heater cartridge
34—Pressure borehole
35a, 35b, 35c—Pressure lines
36—Pressure gauge
37—Pressure actuation valve
38—Pressure safety valve
39—Proportional relief valve

1. A test apparatus 1 for measuring the frictional properties of a fluid, or the properties of a specimen immersed in a fluid, comprising;
a first specimen holder 10 which is adapted to hold a first specimen 13 in the fluid being tested, such that a first specimen surface of the first specimen 13 is in contact with a second specimen surface of a second specimen 14, in a second specimen holder 15, means for applying a measurable load between the two specimens, said test apparatus 1 also comprising oscillatory driving means for oscillating the first specimen holders along a first direction with respect to the second specimen holder, and a force measuring means 24 to measure the frictional force between the two specimens, characterised in that the apparatus 1 includes a sealed pressure chamber 4 in which the first and second specimen holders 10, 15 are located so that the test may be carried out under alternative pressures, and wherein a push rod 9 is provided which connects the oscillatory drive means to the first specimen holder 10 and provides the oscillating motion of the first specimen holder 10 inside the pressure chamber 4 by means of a seal means 18a, 18b so the pressure in the pressure chamber 4 is maintained during the test.

2. A test apparatus 1 according to claim 1, characterised in that a front portion 3 of the apparatus is provided which includes the pressure chamber 4, and which front portion is removable such that the pressure or vacuum is maintained when removed from the apparatus to another location remote from the remainder of the test apparatus 1.

3. A test apparatus 1 according to claim 1, characterised in that the pressure chamber 4 comprises a removable lid 5 with a glass window 6.

4. A test apparatus 1 according to claim 1, characterised in that the pressure chamber 4 comprises an upper chamber part 7 and a lower chamber part 8.

5. A test apparatus 1 according to claim 1, characterised in that the second specimen holder 14 comprises an upstanding wall 16 which contains the test fluid 17 if required.

6. A test apparatus 1 according to claim 1, characterised in that the first specimen holder 10 is located in an upper support block 11 which is attached to seal means in the form of two metal bellows 18a, 18b which extend from each side of the support block 11 and are attached to internal wall 19 of the upper chamber part 7.

7. A test apparatus 1 according to claim 1, characterised in that the push rod 9 is coupled, by means of decouplable push rod coupling 9a to an external vibrator located within a housing 2 which enables the first specimen 13 to be driven in a reciprocating motion.

8. A test apparatus 1 according to claim 1, characterised in that the lower specimen holder 15 is attached to a lower support block 20 which forms part of the lower chamber part 8.

9. A test apparatus 1 according to claim 1, characterised in that two thin discs 21a, 21b are attached to each of respective blocks ends 22a, 22b at all centre region of the discs 21a, 21b, and the discs 21a, 21b being fixed in a circumferential region of the discs 21a, 21b to an inside surface of the lower chamber part 8.

10. A test apparatus 1 according to claim 9, characterised in that the discs 21a, 21b are axially flexible but stiff in radial directions.

11. A test apparatus 1 according to claim 9, characterised in that the discs 21a, 21b act as a flexible seal to allow a pressure gas or vacuum to be maintained inside the chamber 4 on insides of the discs 21a, 21b against an ambient pressure outside the chamber 4 on the outside of the discs 21a, 21b.

12. A test apparatus 1 according to claim 8, characterised in that the lower support block 20 is attached by a pushrod 23 to a force transducer 24.

13. A test apparatus 1 according to claim 12, characterised in that a relative high stiffness of the force transducer 24 to that of the flexible discs 21a, 21b is such that a high proportion of the friction force between the specimens is applied to the force transducer and as the combined discs 21a, 21b and force transducer 24 arrangement is fully elastic a signal from the force transducer 24 is directly proportional to the friction force exerted on the lower specimen 14.

14. A test apparatus 1 according to claim 10, characterised in that the discs 21a, 22b are electrically insulated from the internal wall of the lower chamber part 8.

15. A test apparatus 1 according to claim 1, characterised in that an electrically insulating coupling 27 is provided between pushrod 23 and the force transducer 24 so that the whole transducer arrangement is electrically insulated.

16. A test apparatus 1 according to claim 8, characterised in that the lower specimen holder support block 20 includes a borehole 28 to facilitate the insertion of a temperature measurement probe 29.

17. A test apparatus 1 according to claim 16, characterised in that a heater block 31 is provided containing cartridge heaters 33 to allow the chamber 4 to be heated and the temperature controlled by the input from the temperature measurement probe 29 inputted to a temperature controller 34.

18. A test apparatus 1 according to claim 7, characterised in that the force transducer coupling 27 and the pushrod coupling 9a, the whole pressure chamber test assembly 3 can be removed from the test machine 1 whilst maintaining the pressure in the chamber 4 so that test blends can made within the pressurized chamber 4 away from the remainder of the test machine 1.

19. A test apparatus 1 according to claim 4, characterised in that the lower chamber part 8 includes at least one pressure borehole 34 into which a pressure line 35a, 35b, 35c is fitted.

20. A test apparatus 1 according to claim 19, characterised in that an actuation valve 37 is included in the pressure line 35a, 35b, 35c.

21. (canceled)
